(19) United States
(12) Patent Application Publication
(10) Pub. No.: US 2014/0275843 A1
(43) Pub. Date: Sep. 18, 2014

(54) CONTINUOUS OPTODE FLUORESCENT MEASUREMENT SYSTEM

(71) Applicants: James S. Piccirillo, Middletown, CT (US); Justin Holmes, Deep River, CT (US)

(72) Inventors: James S. Piccirillo, Middletown, CT (US); Justin Holmes, Deep River, CT (US)

(21) Appl. No.: 14/212,509

(22) Filed: Mar. 14, 2014

Related U.S. Application Data
(60) Provisional application No. 61/791,991, filed on Mar. 15, 2013.

(51) Int. Cl. A61B 5/1455; A61B 5/145

(52) U.S. Cl. CPC A61B 5/14556; A61B 5/14532; A61B 5/14546
USPC 600/301; 600/317; 600/316

(57) ABSTRACT

Presently described are systems and methods for the continuous, accurate, low cost measurement of the concentration of target molecule(s), e.g., analytes, such as metabolites and ions, using analyte-specific, light-emitting agents, e.g., fluorescent nanosensors.

### CROSS-REFERENCE TO RELATED APPLICATION

This application claims the benefit under 35 U.S.C. 119(e) of U.S. Provisional Patent Application Ser. No. 61/791,991, titled: Continuous Optode Fluorescent Nanosensor Measurement System, filed Mar. 15, 2013, which is hereby incorporated herein by reference in its entirety for all purposes.

### FIELD OF THE INVENTION

The present disclosure provides, in certain aspects, a system and methods for non-invasively (i.e., minimally invasively), continuous, accurate, low cost detection and measurement of the concentration of target molecule(s), e.g., analytes, such as metabolites and ions in vivo, using an optode and fluorescent nanosensors.

### BACKGROUND

Diabetes is a group of metabolic diseases in which a person has high blood sugar, either because the pancreas does not produce enough insulin, or because cells do not respond to the insulin that is produced. Globally, diabetes is on the rise, and in the United States, it has become a national health-care crisis. The American Diabetes Association recommends that patients with diabetes should try to maintain their glucose levels as close to normal as possible, without causing hypoglycemia. Self-monitoring of glucose is essential for regulation and is often performed through a finger-prick method three times or more per day. However, the need to draw blood, even in small quantities, multiple times a day is not desirable.

Nanosensors and other agents are available and being further developed which selectively absorb one wavelength of light and depending on the concentration of a specific target molecule, linearly modulate the amount of fluorescence at another wavelength of light. The difference between these wavelengths of light is called the Stokes Shift. The nanosensors can be deposited subcutaneously, on or within the skin of a patient in a manner similar to a tattoo, and subsequently used to specifically monitor metabolites or analytes of interest, e.g., glucose.

Thus, the use of fluorescing agents, e.g., nanosensors that specifically recognize an analyte of interest, such as glucose, offer the ability to measure analytes non-invasively; and therefore, are of great commercial interest. Moreover, the introduction of fluorescing (Stokes Shift) nanosensors and their use in temporary skin tattoos craves a low cost personal measurement instrument/system to take advantage of their unique combination.

### SUMMARY

In certain aspects, the present description provides systems comprising optical sensor devices (optode) for accurate measurement of the intensity of fluorescing Stokes Shift nanosensors. In another aspect, the present description provides methods of using devices and systems as described herein. The systems as described herein provide for continuous, accurate, low cost measurement of the concentration of the target molecule(s), e.g., analytes, such as metabolites, electrolytes and/or ions.

Thus, in one embodiment the system comprises at least one analyte-specific, light-emitting agent, e.g., analyte-specific light-emitting nanosensor (collectively, “agent”), a sensor device, and a display, e.g., a hand-held device or computer display, such as a monitor. In certain embodiments, the display is integrated for use with a data website. In additional embodiments, the analyte recognized by the agent is a metabolite, electrolyte or other biomolecule, such as, e.g., glucose, sodium, potassium, calcium, chloride, or a combination thereof.

In certain embodiments, the sensor device comprises at least one light emitting diode (LED), a light detector (i.e., a photodiode) optionally having a light (i.e., wavelength) filter, and a transceiver. In additional embodiments, the sensor comprises an SOC/microprocessor, encrypted memory or both. The sensor is designed to be near or in apposition to (e.g., held or fixed in place by, for example, a strap, belt or the like) the surface of the skin of a subject, such as a patient. In certain embodiments, the sensor is reversibly attachable to the surface of the skin of a subject. In additional embodiments, the sensor is disposed or contained in a housing, which can is convenient for wearing or holding. In certain additional embodiments, the sensor device comprises an additional sensor capable of monitoring, by wire or wirelessly, a subject's vital signs, such as heart rate, blood pressure, temperature or the like.

In certain embodiments, the system is configured to acquire continuous, real-time measurement of level or concentration of an analyte of interest. In additional embodiments, the system is configured such that the sensor quantifies or calculates the in vivo amount or concentration of the analyte and transmits the information to the display, e.g., of a hand-held device or computer.

In a preferred mode of operation, the system is configured with appropriate nanosensors to quantify the in vivo levels or concentration of a desired analyte, such as a metabolite or electrolyte, e.g., glucose, sodium, potassium, calcium, chloride, or other molecule or ion, in a sample, e.g., a biological sample or tissue of a subject. In certain embodiments, the system may be configured to acquire continuous, real-time measurement of the analyte concentrations at a predetermined rate of acquisition, e.g., every fraction of a second, second, minute, hour, day(s), weeks, months, etc. or combination thereof. In additional embodiments of the system, the data is communicated, e.g., electronically communicated wirelessly to a device (e.g., hand-held device or computer) which can display the results, e.g., analyte levels, concentration, vital signs, battery level, etc. or the like, and/or transfers the data to a processor having a display, e.g., Internet Website, and processor for long term storage and advanced analysis. The data can also be securely shared between the user and other personnel.

In another aspect, the description provides methods of using the systems as described herein. In certain embodiments, the methods may comprise a step wherein the analyte level or concentration data is used by a health professional to diagnose, monitor, and/or treat a disease or disorder.

In another aspect, the description provides a system as described herein that further comprises a drug delivery device, e.g., electronic pump or the like, which is in communication with the sensor and/or the interface display. In certain embodiments, the sensor communicates with the drug delivery device in a closed-loop feedback system to modulate the activity of the drug delivery device, e.g., increasing, decreasing, maintaining, or ceasing the delivery of a therapeutic agent, e.g., insulin (in the case of a treatment for diabetes) or other medication, in response to analyte measurements determined by the sensor.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages, objects, and embodiments are expressly included within the scope of the present invention. The publications and other materials used herein to illuminate the background of the invention, and in particular cases, to provide additional details respecting the practice, are incorporated by reference, and for convenience are listed in the appended bibliography.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1. Schematic diagram of an exemplary system as described and shown herein.

FIG. 2. Photo of exemplary prototype system having an LED Printed Circuit Board Assembly (PCBA) 210 which contains hundreds of 460 nm LEDs and 570 nm LEDs. The 570 nm LEDs are illuminated. These LEDs simulate the fluorescing wavelength of the nanosensors. On the lower left is the light box which is used to simulate skin and the nanosensor tattoo. In the skin, the 460 nm wavelength of light is used to excite the nanosensors to fluoresce at 570 nm. The 12″×12″×0.75″ Lexan block 220 is frosted on the top so that a large number of sensors can be placed on the surface and each detector would “see” the same illumination.

FIG. 3. Photo of exemplary prototype system having an LED Printed Circuit Board Assembly (PCBA) 310 which contains hundreds of 460 nm LEDs and 570 nm LEDs. In this figure the 460 nm LEDs are illuminated to simulate the emission of light by the LEDs located on the bottom of the sensor (i.e., light to excite the nanosensor). Lexan block 320 is frosted on the top so that a large number of sensors can be placed on the surface and each detector would “see” the same illumination.

FIG. 4. Photo showing the exemplary prototype system having an LED PCBA 410 (4) with the 570 nm LEDs illuminated. When the 4 LED PCBAs are mounted onto the sides of the Lexan block 420, most of the illumination from the LEDs is launched directly (<critical angle) into the polished side surfaces of the Lexan block. The LED light is captured within the block and randomly bounces off the interior block walls. The frosted top surface allows some light to escape and this illuminates the surface very evenly. The intensity of the light from the two different LED wavelengths is measured by the down ward looking detector on the sensor. There are two examples of Kodak filters 430 on the surface of the block.

FIG. 5. Photo showing the exemplary prototype system having an LED PCBA (4) with the 460 nm LEDs 510 illuminated. The 4 LED PCBAs are mounted onto the sides of the Lexan block 520. There are two examples of Kodak filters 530 on the surface of the block.

FIG. 6. Photo of top side of exemplary sensor PCBA 640. The sensor comprises a detector. This is the top of the sensor PCBA 640 (without SOC). Photo also shows connector pins for cable to SOC exemplary PCBA. There are two examples of Kodak filters 630 on the surface of the Lexan block 620.

FIG. 7. Photo of bottom side or “skin side” of the exemplary sensor PCBA 740 showing the window of the detector 750 (black square in the center). The sensor PCBA 740 also comprises LED lights mounted on the bottom of the sensor PCBA proximal to the window of the detector 750 (in this example the two LEDs are 460 nm). It is preferred to locate the LEDs as close to the window of the detector as possible so that the detector receives sufficient emitted light from the nanosensor(s). In certain embodiments, a light (i.e., wavelength) filter is disposed over the window of the detector 750. There are two examples of Kodak filters 730 on the surface of the Lexan block 720. The LED PCBA 710 is also shown.

FIG. 8. Photo of exemplary sensor 840 from FIG. 7. The window of the detector 850 is the small black square in the middle of the PCBA. In this example, the sensor PCBA 840 comprises LEDs 860 (in this example the two LEDs are 460 nm) on either side of the window of the detector 850. It is contemplated that the sensor PCBA can comprise a plurality of LEDs of different emission wavelengths, e.g., to correspond to the excitation wavelengths of the respective nanosensors employed.

FIG. 9. Photo of exemplary integrated sensor PCBA 940. The sensor system comprises a sensor PCBA 940 comprising LEDs (not shown), a detector (not shown), a filter (not shown), a battery 905 and an OpAmp. The sensor is attached to an analog-to-digital converter (ADC) 960 via cable connector 970. The exemplary PCBA on the left (960) comprises an SOC, microprocessor, encrypted memory, and transceiver. The integrated sensor is shown resting on the Lexan block 920 with the LED PCBAs 910.

FIG. 10. Shows components of an exemplary device as described herein. The quarter on the left is provided for scale. In particular, the figure shows a cap 10-01 (i.e., top) of the exemplary device housing. The center of the figure shows the top (i.e., upward facing portion) of a sensor printed circuit board assembly (PCBA) 10-40 comprising a printed circuit board 10-41, a battery 10-05, microprocessor 10-06, and RF Blue Tooth Antenna 10-07 (small silver rectangle). In a preferred embodiment the PCBA 10-40 is secured within a housing that allows for emission and detection of light. The microprocessor 10-06 and Blue Tooth Antenna 10-07 measure the light emitted and detected, and transmit the light intensity data to a device, e.g., a mobile device, respectively. The PCBA 10-40 may be mounted within the housing via through-holes 10-04.

FIG. 11. Shows the bottom of the PCBA of FIG. 10 (11-40). Right and Left items are the same as before. At the center of the PCBA 11-40 is the window of the 570 nm detector 11-50 and LEDs 11-60, which are affixed to the PCBA 11-40. The PCBA 11-40 is mounted within the housing via through-holes 11-04.

FIG. 12. Shows the top 12-01 and bottom 12-08 of the housing of the exemplary device comprising the PCBA as described in FIGS. 10 and 11. Visible are the window of the detector 12-50, LEDs 12-60, and through-holes 12-04 (containing screws) for mounting the bottom 12-08 of the housing to the top 12-01. The PCBA components (12-50, 12-60) are visible through a clear covering 12-09 in the housing (in certain embodiments, a light filter.

FIG. 13. Illustrates a circuit diagram for an exemplary device as described herein that is configured for constant current to LEDs.

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

All numerical values within the detailed description and the claims herein are modified by “about” or “approximately” the indicated value, and take into account experimental error and variations that would be expected by a person having ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. Ranges from any lower limit to any upper limit are contemplated. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

“Analyte” as used herein can mean but is in no way limited to a biomolecule, component, substance or chemical constituent that is of interest in an analytical procedure and is to be measured or detected. For example, in certain embodiments, the analyte is glucose and the property to be measured using the devices and systems provided herein, is glucose concentration.

In certain aspects, the present description provides systems comprising optical sensor devices (optode) for accurate measurement of the intensity of fluorescing Stokes Shift nanosensors. In another aspect, the present description provides methods of using devices and systems as described herein. The systems as described herein provide for non-invasive (i.e., minimally invasive) continuous, accurate, low cost measurement of the levels and/or concentration of the target molecule(s), i.e., analytes, such as, e.g., metabolites, electrolytes, and ions.

As such, in certain embodiments, the description provides an optical sensor (optode) system comprising at least one analyte-specific, light-emitting agent, which emits light in the presence of the analyte; a sensor device configured to detect non-invasively the light emitted by the agent, the sensor device comprising a plurality of LEDs, at least one photodiode configured to detect light emitted from the light-emitting agent, an SOC/microprocessor, and transceiver to receive/transmit data wirelessly and, optionally, a photodiode light filter; and a display, wherein the system is configured such that the sensor device quantifies the in vivo levels or concentration of the analyte and displays the information graphically on the display. In certain embodiments, the system is configured to acquire continuous real-time measurement of the analyte concentration

In certain additional embodiments, the system of comprises two LEDs. In yet other embodiments, the LEDs are located near or in apposition to the photodiode or window thereof. In certain additional embodiments, the sensor device further comprises an OpAmp, an ADC, an encrypted memory or a combination thereof.

In additional embodiments, the system comprises a display such as a hand-held device display or a computer monitor. In certain embodiment, the display is integrated with a data website.

In certain embodiments, the analyte to be measure is selected from the group consisting of glucose, sodium, potassium, calcium, chloride, and a combination thereof.

In another aspect, the description provides a system comprising a sensor device as described herein, and a drug delivery device in communication with the sensor device.

In certain additional aspects, the description provides a sensor device as described herein, which comprises at least one additional sensor element that detects and/or measures a vital sign in a subject, e.g., blood pressure, heart rate, temperature, etc. or a combination thereof.

Exemplary components of the system are described further below. The examples described herein are intended to illustrate rather than limit the invention. As will be appreciated by the skilled artisan, the components of the system can be optimized and combined in a number of ways, all of which are expressly contemplated by the inventors.

Nanosensors

In certain embodiments, the system comprises at least one analyte specific, light-emitting nanosensor, a sensor device, and a display, e.g., on a hand-held device or computer monitor. As indicated above, the analyte can be any biomolecule of interest, e.g., glucose, sodium, potassium, calcium, chloride, or a combination thereof. Specially designed nanosensors will fluoresce at different intensities in the presence of a target molecule, such as glucose. Due to the bio-chemical equilibrium of these nanosensors, a higher glucose concentration results in a lower intensity of the Stokes shifted wavelength of light. Certain nanosensors suitable for use in the devices and systems as described herein are described in Balaconis et al., Biodegradable optode-based nanosensors for in vivo monitoring. Anal Chem. 2012 Jul. 3; 84(13):5787-93, which is hereby incorporated by reference in its entirety. For example, a suitable glucose-specific fluorescent agent includes FLIPglu-600 μM, glucose oxidase (GOx) based electrochemical biosensors, derivatives and analogs thereof. Additional nanosensors suitable for use in the present system and methods include, copper and copper oxide nanowires, porous films as well as nanoflowers and nanorods, nanostructured copper oxide/copper oxalate, nanoparticles composed of silver, gold, nickel, and nickel/palladium, such as gold nanowires, nickel hydroxide nanocomposites, boron-doped diamond nanorods, platinum/lead nanoporous networks, palladium nanoparticles, fluorescent polymeric nanosensors. See, e.g., Cash and Clark, Trends Mol Med. Sep. 23, 2010; 16(12): 584-593, which is incorporated herein by reference.

It has been shown that specific designed nanosensors can be applied on, or injected into skin (e.g., tattooed) and leave no visible mark. Skin is nearly transparent at the infrared wavelengths of light used with these types of nanosensors. The nanosensor tattoo is intra-dermal and generally lasts about one week. After a week, the superficial layers of a human's skin would be sloughed off along with the nanosensor tattoo. This would require the re-application of the nanosensor tattoo and at the same time allow a new sensor with battery or the reuse of a sensor with a freshly recharged battery to be applied to the skin.

A nanosensor tattoo can be applied in any desired area and be of any suitable size, e.g., from 10 cm² to 0.1 mm², and then a sensor can be attached over this area. In a preferred embodiment, the nanosensor tattoo will be of a size that corresponds to the detection window of the sensor device, which should include sufficient area for the exposed LEDs and photodiode surfaces. In certain embodiments, the tattoo and the sensor can be applied to the skin in one step. It should be understood that it is important that the sensor device (comprising the LEDs and photodiode) be placed over the location of the nanosensor tattoo. Ambient light and the excitation wavelength of light must be prevented from reaching the photodiode (e.g., 570 nm light detector) because it can cause measurement errors. In certain embodiments, the sensor device may be detachably fixed or secured to the skin by, e.g., straps, glue, tape or any other method known to those of skill in the art.

Nanosensors which target different molecules and are excited by different wavelengths of light can be used in the same tattoo. Thus, in certain aspects, the disclosure provides a dermal or subdermal injection or tattoo comprising one or more analyte-sensitive nanosensors. In a preferred embodiment, each nanosensor has a different emission wavelength. In still another preferred embodiment, each nanosensor has a different excitation and emission wavelength. By designing the nanosensors with this feature and if they have the same fluorescing wavelength of light the real time measurement of different target molecules/ions can be obtained with one detector (saving cost). By turning “on” and “off” selected LEDs with different excitation wavelengths of light, the concentration of different target molecules/ions can be quantified at different times or at approximately the same time. As an added advantage, this allows charting of different measured values at the same time.

Sensor

After the appropriate nanosensors are implanted into the skin, the sensor unit is placed over the “tattooed” area. The sensor may be taped or adhered to the small sensing area. Because the sensor devices as described herein are inexpensive to make, in certain embodiments, the sensor device is disposable and can be discarded when the active life spans of the nanosensors have expired. Alternatively, a new tattoo could be applied and the battery of the sensor can be replaced or recharged. In certain embodiments, the application method of the temporary tattoo can be part of attaching the sensor onto the skin of the user. In certain embodiments, the nanosensor(s) can be placed dermally, sub-dermally or intra-dermally. As the skilled artisan will appreciate, the precise location of the nanosensor tattoo depends on the type or types of analyte to be measured.

The sensor devices as described herein perform several functions at very low cost and consume very low power. In certain embodiments, the sensor comprises one or more of the following components: a light source, e.g., at least one LED, a light filter, a photodiode or light detector, electronics to scale the output voltage/current of the detector, an analog-to-digital converter, a microprocessor, encrypted memory, RF transceiver, a battery, and combinations thereof. In a preferred embodiment, the sensor comprises a light source, light filter, detector, electronics to scale the output voltage/current of the detector, an analog-to-digital converter, microprocessor, encrypted memory, RF transceiver, and a battery.

A light source must be selected so that it provides the correct excitation wavelength of light for specific nanosensors. For example, in the case of a glucometer, the source wavelength is preferably 460 nm. Exemplary light sources comprise one or more LEDs, e.g., for glucose, 460 nm LEDs. In order to conserve battery power, these LEDs can be configured such that they are only powered when samples of the fluorescing nanosensors are being collected. Frequently, the voltage needed to illuminate the source LEDs is greater than the battery voltage so a voltage doubling electronic circuit (or more) may be used. In certain instances, the intensity of the LEDs may vary over its use and its lifetime. The change in the intensity of the LEDs can thus be characterized mathematically and the collected data modified accordingly to minimize errors.

In certain embodiments, the sensor device comprises an ultra-low power operation amplifier (OpAmp), which is used to scale the output signal of the detector (may be contained within the SOC (System-On-Chip). In an exemplary device, A TI CC2540/41 (See FIG. 8) was used. The low noise OpAmp scales the sensor output current to a usable voltage level of 0-2 Volts DC. This area of circuitry is configured to minimize electrical noise to the signal being measured. Therefore, accurate measurement of very small changes of the fluorescing wavelength of light is possible. With a 12 bit ADC measuring a 0-2 volt signal, each bit represents 0.000488 volts per bit (2 volts/4096). Note that 488 micro volts is a very small voltage so as much electrical noise must be minimize. Electrical noise causes an error in the signal voltage which is being determined. This includes using the shortest possible signal PCB runs, shielding runs, and crate “circuit guards” to provide the most rejection of interference to the signal being measured.

In certain embodiments, the sensor device comprises at least one of a light emitting diode (LED), a light detector (i.e., a photodiode), a light (i.e., wavelength) filter, a transceiver or a combination thereof. In a preferred embodiment, the LEDs are located proximal to the window of the photodiode to as to maximize the amount of light emitted by the nanosensors in the area below the detector. It is contemplated that the sensor PCBA can comprise a plurality of LEDs of different emission wavelengths, e.g., to correspond to the excitation wavelengths of the respective nanosensors employed.

In additional embodiments, the sensor device also comprises an opamp, a microprocessor, memory, a power source or a combination thereof. In certain embodiments, the sensor may additionally comprise a transceiver (e.g., wired or wireless). In certain embodiments, the sensor is reversibly attachable to the surface of the skin of a subject. In additional embodiments, the sensor is comprised or housed in a hand-held monitoring device. In certain additional embodiments, the sensor comprises an additional sensor capable of monitoring, by wire or wirelessly, a subject's vital signs, such as heart rate, blood pressure, temperature or the like.

Photodiode

In a preferred embodiment, the sensor device measures the intensity of 570 nm light, and attenuates the 460 nm light. The photodiode is more sensitive to the 570 nm but the 460 nm signal still creates a large error and must be attenuated. There are several optical filters which can be used. In the exemplary embodiment, a low cost filter, e.g., a multilayer Kodak Wratten Filter #12 (See, e.g., FIG. 4, 440; FIG. 5, 540) can be used. The use of this filter resulted in a >90% attenuation of the 460 nm in the presence of the 570 nm signal.

Photodiodes suitable for use with devices and systems as described herein, are commercially available, e.g., an Osram SFH2430 photodiode, which was used in the exemplary device. The output of the photodiode is a current which is proportionate to the amount of light striking the surface of the detector. In this case, the photodiode is most sensitive to light at the frequency of 570 nm.

Battery Power of Sensor

Because of the ultra-low power consumption of the sensor and control device, in certain embodiments, small coin cell batteries can be used as the power source. These coin cell batteries can be two different types, one time use and rechargeable. The “throw away” batteries are disposed of when their power is depleted. As an alternative, rechargeable batteries can also be designed into the sensor and controller. If the device uses a large capacity coin cell battery (i.e. CR2035) the operation of the sensor and controller will be a year or more. On the other hand, a smaller battery, sensor, and controller could be over molded and thrown away after the battery is depleted (or possibly recharged). Recharging could be done without wire contacts if an AC signal is used as the power source (similar to rechargers for popular electric tooth brushes).

Sensor Microprocessor

In the exemplary embodiments of the Figures, the sensor device, including the LEDs, are controlled by a microprocessor, e.g., a Texas Instrument (TI) System-On-Chip (SOC). The microprocessor controls the sensor operation (ADC data output), raw data storage in memory, and the transceiver communication back and forth to the Hand Held Device. The microprocessor and the elements of the sensor can be very small. Together they could be smaller than the diameter of a dime. The sample rate could be set to a low as one sample per second or lower to thousands per second depending on the requirements.

Hand Held Device Algorithmic Processing of ADC Data

Algorithmic methods for increasing the resolution of the ADC beyond the default bit resolution (i.e. 12 bit to 13 bit or more) can be used because the equilibrium state of the nanosensors follows the slow changes of the analyte. As an example, blood glucose concentrations are normally measured with a “finger prick” before meals, at bed time, and other times. The level of blood glucose changes between the individual data points. In general terms these levels change over hours of time. This technique is very discontinuous and results in a low understanding of blood sugar short term trends. Also, there is no ability to establish an understanding of long term trends.

This solution intends to measure a variety of molecule/ion concentrations. Nanosensor tattoos have been developed to measure many different molecule/ion concentrations. As an example, this system measures blood sugar glucose concentrations every minute. At the start of each minute measurement samples are taken for a short period of time (i.e. one second). Mutable ADC readings are taken during this sample period. The large number of ADC values are then used to mathematically resolve extended bits of resolution. There are several mathematical methods which can be performed known to those of skill in the art. In general, this is called extending the precision of the ADC.

Extending the ADC Bit Resolution

Multiple samples are taken at 12 bit resolution. In this case, the ADC values dither between two adjacent values. There are a total of 101 samples taken during the sample period (i.e. one second). If there are more that 51 samples of the larger value ADC sample then the extended bit would be set to “1”. If there are more that 51 samples of the smaller value ADC sample then the extended bit would be set to “0”. In order for this case to work, one has to always use an odd number of samples and the values must dither between two adjacent ADC values. This can be extended to values changing between more than two adjacent ADC values. Another method to extend the ADC bit resolution is doing sufficient samples to obtain a Gaussian curve. The calculated value is the point when the slope of the Gaussian curve is zero.

Sensor Memory and Hand Held Device Memory

In certain embodiments, the system provides secure encrypted communication between the sensor device and the Hand Held Device. In additional embodiments, the system provides secure encrypted wireless communication between the Hand Held Device and a Website. Since these links can be transient each element must have sufficient data memory to queue the data until it can be deterministically transferred to the Website for permanent storage and availability. In additional embodiments, the user can request long term data from the website database to be displayed on the hand held device for review on a chart. In certain embodiments, the sensor device comprises ports for direct wired connection to other devices allowing display or transfer of data.

Charting of Data

The hand held device and website will have the same look and feel for charts, data, and calibration data points. The user should be able to easily switch between the Website and the Hand Held Device and operate them similarly.

In general, there are many ways to display and analyze the data provided by the system. In a preferred embodiment, the hand held device display and website have the same look and feel for charts, data, and calibration data points. In certain embodiments, the system is configured such that multiple users can acces the same respective data and independently customize the display for each users particular use. In additional embodiments, the individuation of the display can be defined when each person logs into the website.

In certain embodiments, the basic graphic user interface (GUI) is divided into multiple windows, e.g., each showing a different measured parameter, such as battery voltage, blood glucose, pulse, etc. In certain embodiments, the display comprises a toggle or drop down menu listing the name of the measured parameter, which can be independently selected by the user. In yet additional embodiments, the name of the parameter measured is followed by the last value read from the sensor (i.e. 2.97 VDC or 127 mg/dL). In additional embodiments, if the value is touched by the user it can be changed to an alternate unit of measurement or other statistical indicia, e.g., the 24 hour high value or the 24 hour low value. In certain embodiments, the display also comprises an up or down arrow (optionally with color differentiating color) showing the direction of the last values displayed compared to the previous data point value. In additional embodiments, the color of the button or parameter name selected would change if attention is required (i.e. battery voltage is low or BG is high or low).

In additional embodiments, the display also comprises a chart of the data collected by the selected sensor. In certain embodiments, on one axis the chart indicates the time value, e.g., 1 second, 1 minute, 1 hour, 1 day, 1 week, 1 month, 1 year, 2 years, etc. In still additional embodiments, by pressing on one of these values the corresponding data values are displayed on the chart. In additional embodiments, the other axis indicates the values of the measured parameters. In still additional embodiments, the graphical data obtained from the sensor device can be transmit to a hand held device or up loaded to a website database.

Communication of Data Between Hand Held Device and Website

The same collected data is available for viewing on the Hand Held Device and the Website. The user can select different lengths of time (i.e. day, week, etc.) and the data collected during the time period is displayed in the chart. Data can move back and forth between Hand Held Device and the Website. The raw data can be stored on the Sensor if the Hand Held Device is not available.

Chart Data Calibration

There are many ways to calibrate the data displayed on the chart. For example, calibration may be completed during the manufacturing process of the nanosensors. In certain embodiments, a calibration curve is created for each batch of nanosensors when they are manufactured. A number is assigned to each calibration curve and this number is used by the hand held device when a sensor device is attached to the nanosensor tattoo.

In addition to this method, the data on the chart can be calibrated by using a known standard and imputing the data into the chart as a data point with a unique icon. The chart values can then be adjusted appropriately. As a glucose data point example, a “finger stick” could provide a sample of blood and using a glucometer a blood glucose value could be determined. This data point is then entered into the chart on the hand held device or website. In certain embodiments, the glucomter data point is used to calibrate the values measured by the sensor device, and displayed in the chart. Also, the hand held device could prompt the user to provide a calibration sample at the appropriate times.

Icons with comments can also be added the chart. Comments such as the user's amount of physical activity, weight, use of medications, etc.

The chart data can be configured using known standardizing algorithms. The calibration data can also be communicated to the handheld device by wire or wirelessly.

Exemplary systems as provided by the present disclosure are described further below, with certain reference to the exemplary embodiments depicted in the drawings. The examples as described and shown herein are intended to illustrate rather than limit the invention. As will be appreciated by the skilled artisan, the system can be optimized and varied in a number of ways, all of which are expressly contemplated by the inventors.

### EXAMPLES

FIG. 1 shows a schematic diagram of an exemplary system as described and shown herein. An nanosensor tattoo comprising one or more analyte specific nanosensors is placed or located below the surface of the skin of a patient, for example, below the epidermis. In a preferred embodiment, the nanosensor tattoo is placed above the hypodermis. In still another preferred embodiment, the ananosensor tattoo is placed approximately within the dermal layer.

To measure the analyte(s), a sensor device is placed approximately in apposition to the surface of the skin above the location of the nanosensor tattoo. In certain exemplary embodiments, the sensor includes a light source, e.g., LED, a photodiode selected to respond to the Stokes shifted wavelength of light and optionally including a filter, and OpAmp, an SOC/microprocessor, encrypted memory for data storage and operational instructions to control the microprocessor, and RF or wired transceiver for transmitting data to a hand held device or processor or a combination thereof. In certain embodiments, the hand held device or processor comprises a display (i.e., graphic user interface). In certain embodiments, the display is an internet web site or a hand held device application (“app”) or both. In additional embodiments, the display allows for visualization of the analyte measurements, data analysis, and access by one or more users (e.g., patient and care provider).

In certain additional embodiments, the sensor device comprises at least one wireless sensor capable of detecting a subject's vital signs, e.g., heart rate, blood pressure, temperature. In such instances, the interface display (i.e., graphic user interface) is configured to display the vital sign information as well.

In certain additional embodiments, the sensor also transmits information regarding the amount of charge remaining in the sensor batteries. In such instances, the interface display (i.e., graphic user interface) is configured to display the battery life information as well.

For example, the wavelength of light used to excite the nanosensors is 460 nm. Depending on the concentration of glucose, an inversely proportional amount of 570 nm wavelength of light is produced. The system as described herein measures and scales measured data such that it represents the amount of fluorescing wavelength light created by these nanosensors.

In order to design and test an exemplary system as described herein, both wavelengths of light were created and accurately controlled in a test bed light box. The wavelengths of the light sources used in the light box were 460 nm and 570 nm. The 460 nm wavelength of light excites the nanosensors and they fluoresce at a wavelength of 570 nm. So, the intensity of the 570 nm wavelength of light is the only wavelength of light which should be quantified; any other wavelength of light which reaches the detector causes an error in the value measured. In the testing light box both wavelengths of light must be present and controlled. Light emitting diodes (LEDs) of each wavelength were purchased and a test bed light box for sensor development and system evaluation was created.

Certain exemplary systems comprise several components (in addition to the nanosensor tattoo). In certain embodiments the system is configured to comprise at least one of a detector, a light source, a selected wavelength light filter, a photodiode, an OpAmp, a microprocessor, memory, a power source (e.g., coin cell battery rechargeable or non-rechargeable) or a combination thereof. In certain embodiments, the sensor may additionally comprise a transceiver (e.g., wired or wireless). In certain additional embodiments, the sensor is configured or housed within a handheld device (e.g., an iPhone, iPod, iPad, smart phone, or the like).

In certain embodiments, the system includes a first transceiver configured to communicate to the sensor (e.g., via wire or wirelessly (i.e. BT, BLE)), a microprocessor, encrypted memory, a second transceiver configured to communicate to the Internet via wire (i.e. Ethernet) or wirelessly (i.e. BT, BLE, Wi-Fi, telephony, cell phone)), and the secure Website (iHALP.net).

In certain embodiments, for example, a continuous glucose concentration monitoring system, the user can customize the parameters to be displayed by the hand held device and/or computer display, e.g., internet website. For example, the user can setup alarms to notify the user when certain glucose concentrations are detected, e.g., “High Glucose ml/dL” and “Low Glucose ml/dL”. Different indicia can be selected by the user, such as mmol/L. As stated before, different molecule concentrations could be measured depending on the type of nanosensor selected. In additional embodiments, multiple indicia can be displayed approximately contemporaneously.

In certain additional embodiments, the system includes a program designed to run on a hand held device and/or a website that can graphically display charts on the short term and long term analyte measurement trends. In certain preferred embodiments, data is collected by the system periodically, e.g., every 0.001 s, 0.01 s, 0.1 s, 1 s, 2 s, 3 s, 4 s, 5 s, 6 s, 7 s, 8 s, 9 s, 10 s, 30 s, 60 s, minute, hour, day, week, month, year(s) or combination thereof, and displayed on a chart. In a preferred embodiment, the data on the hand held device can travel in two directions, from the hand held device to the website and back to the hand held device as needed. In one embodiment, a chart can be displayed by filling the screen from right to left by selecting the length of time 1 hour, 1 day, 1 week, 1 month, 3 months, 1 year and more. Also, by using the historical data stored on the website, long term trends could be used to calculate things like 3 month averages such as hemoglobin A1C.

Also, calibration or reference blood glucose levels can be entered directly into the charts. As an example, the user could enter results of an individual “finger stick” from an off-the-shelf glucometer. The end user could then enter this data into the Website chart or the hand held device chart. This data could be used to calibrate the continuous blood glucose concentrations readings from the optical sensor.

The device illustrated in FIGS. 2-5 represent a proof-of-concept in which certain elements of a device as described herein are configured. For example, FIGS. 2-3 show an exemplary prototype, which demonstrates the functioning of the system. In particular, FIGS. 2 and 3 show LED PCBAs 210, 310, respectively. Each LED PCBA has 460 nm LED lights 310 and 570 nm LED lights 210. It can be appreciated that the light sources may contain only a single wavelength LED or a mixture of two or more, e.g., 460 nm and 570 nm LED lights, or other desired wavelength LEDs. The Lexan block 220, 320 is used to allow for even light distribution across the surface, approximating the operation of the system on skin.

FIGS. 4-5 further show an exemplary or prototype system as depicted in FIGS. 2 and 3, including the Lexan block (420 and 520, respectively), and LED PCBAs (410 and 510, respectively). The light from the sides of the polished Lexan block (420 and 520) bounce around within the Lexan block. The frosted top surface allows some light to escape. The intensity of the light is then able to be measured for testing purposes. The intensity of each wavelength is very even over the whole surface of the block. There are two Kodak filters (430 and 530, respectively) on the surface to allow only selected wavelengths to be visible to the sensor as described and shown below.

FIG. 6 is a top perspective view of an exemplary sensor PCBA 640, including a battery 605. The Lexan block 620 and filters 630 are also shown. FIG. 7 shows the bottom perspective view of the exemplary sensor of FIG. 6. The window of the detector or photodiode 750 is in the center of the sensor PCBA 740. The sensor PCBA 740 also comprises LED lights 760 mounted on the bottom of the sensor PCBA 740 proximal to the window of the detector or photodiode 750 (in this example the two LEDs are 460 nm). The Lexan block 720 and filters 730 are also shown. In a preferred embodiment, the LEDs emit light into or through the top layer(s) of skin of a subject (represented by the Lexan block having a frosted top surface). The light emitted by the LED is at a wavelength suitable for exciting the particular nanosensor. Therefore, it is desirable to configure the LEDs as close to the window of the detector as possible. It is contemplated that the sensor PCBA can comprise a plurality of LEDs of different emission wavelengths, e.g., to correspond to the excitation wavelengths of the respective nanosensors employed.

Referring now to FIG. 8, showing bottom plan view (skin facing side) of the exemplary sensor PCBA 840 of FIGS. 6 and 7. The LEDs 860 emit light, which passes through the window of the detector or photodiode 850. In certain embodiments, a light filter or combination of light filters is disposed on the surface of the detector window. In certain embodiments, the window of the detector or photodiode 850 is centrally disposed along the sensor PCBA 840 although the invention is not so limited. However, it is desirable that the detector or window of the detector be positioned proximally (i.e., juxtaposed, in apposition to or near) to the LED light source in order to maximize the amount of emitted light from the excited nanosensors that is detected by the detector.

FIG. 9 shows an exemplary assembly as described herein. In this example, sensor PCBA 940 comprises a detector, filter and OpAmp and is in electrical communication via cable connectors 970 with an analog to digital converter (ADC) 960. The sensor PCBA 940 sends data over the electrical connection to the ADC for further processing and storage. The sensor PCBA 940 output is scaled for 0-2 VDC output and fed to the ADC 960 for signal processing and wireless transmission to a hand held device (not shown).

FIG. 10 shows components of an exemplary device as described herein. The quarter on the left is provided for scale. In particular, the figure shows a cap 10-01 (i.e., top) of the exemplary device housing. The center of the figure shows the top (i.e., upward facing portion) of a sensor printed circuit board assembly (PCBA) 10-40 comprising a printed circuit board 10-41, a battery 10-05, microprocessor 10-06, and RF Blue Tooth Antenna 10-07 (small silver rectangle). In a preferred embodiment the PCBA 10-40 is secured within a housing that allows for emission and detection of light. The microprocessor 10-06 and Blue Tooth Antenna 10-07 measure the light emitted and detected, and transmit the light intensity data to a device, e.g., a mobile device, respectively. The PCBA 10-40 may be mounted within the housing via through-holes 10-04.

FIG. 11 shows the bottom of the PCBA of FIG. 10 (11-40). Right and Left items are the same as before. At the center of the PCBA 11-40 is the window of the 570 nm detector or photodiode 11-50 and LEDs 11-60, which are affixed to the PCBA 11-40. The PCBA 11-40 is then mounted within the housing via through-holes 11-04.

FIG. 12 shows the top 12-01 and bottom 12-08 of the housing of the exemplary device comprising the PCBA as described in FIGS. 10 and 11. Visible are the window of the detector or photodiode 12-50, LEDs 12-60, and through-holes 12-04 (containing screws) for mounting the bottom 12-08 of the housing to the top 12-01. The PCBA components (12-50, 12-60) are visible through a clear covering 12-09 in the housing (e.g., 500 nm filter) can be placed over the photodiode between the photodiode surface and the clear covering 12-09, which can be lexan).

In certain embodiments, the system is configured to acquire continuous real-time measurement of the analyte level or concentration. In additional embodiments, the system is configured such that the sensor quantifies the in vivo levels or concentration of the analyte and transmits the information to the interface display, e.g., on a hand-held device or computer.

In a preferred mode of operation, the system is configured with appropriate nanosensors to quantify the in-vivo levels or concentration of a desired analyte, such as a metabolite or electrolyte, e.g., glucose, sodium, potassium, calcium, chloride, or other molecule or ion, in a sample, e.g., a biological sample or tissue of a subject. In certain embodiments, the system may be configured to acquire continuous real-time measurement of the analyte concentrations. In additional embodiments of the system, the data is communicated, e.g., wirelessly communicated, to a device (e.g., hand-held device or computer) which can display the results, e.g., analyte levels, concentration, vital signs, battery level, etc. or the like, and/or transfers the data to a processor having a display, e.g., Internet Website, and processor for long term storage and advanced analysis. The data can also be securely shared between the user and other personnel.

In another aspect, the description provides methods of using the systems as described herein. In certain embodiments, the methods may comprise a step wherein the analyte level or concentration data is used by a health professional to diagnose, monitor, and/or treat a disease or disorder.

In a preferred mode of operation, the system is configured with appropriate nanosensors to quantify the in-vivo levels or concentration of a desired analyte, e.g., glucose, sodium, potassium, calcium, chloride, or other molecule or ion, in a sample, e.g., a biological sample or tissue of a subject. In certain embodiments, the system may be configured to acquire continuous real-time measurement of the molecule/ion concentrations. In additional embodiments of the system, the data is communicated, e.g., wirelessly communicated, to a hand held device having a display and/or which then transfers the data to a computer having a display, e.g., Internet Website, and/or a processor for long term storage and advanced analysis. The data can also be securely shared between the user and other personnel.

Open Loop vs. Closed Loop System

The system previously described is an open loop control system; there is no feedback signal/method. The system may require human intervention to analyze the values of the real time data and then perform the appropriate medical action.

A mechanical pump (or other device) which delivers precise variable dosages of medication can be included that uses the collected data to calculate the dosage for use in a closed-loop control system.

As an example of a closed-loop system application, glucose concentration may be measured by the system and depending on a mathematical algorithm, the closed-loop system calculates the appropriate insulin dosage to be dispensed. With the added ability to measure multiple biological parameters (heart rate, respiration rate, blood pressure, other ion concentrations, etc.) the algorithm may take these factors into account (short and long term) and adjust the medication dosage to the appropriate rate on a minute by minute basis. The algorithm can also be configured to adjust dosage depending on previous historical data values (hysteresis).

For example, it is known that a young type 1 diabetic child, using a Medtronic Mini-Med insulin pump (capable of dispensing volumes of insulin as small as 0.001 ml), could be given a precise bolus of Insulin by a parent using a wireless device communicating to the pump. The blood glucose concentration is determined by a glucose meter “finger stick” and then the appropriate insulin dosage is assessed by the parent and then transmitted to the pump to be administered.

Thus, in another aspect, the system comprises a drug delivery device, e.g., electronic pump or the like, which is in communication with the sensor and/or the interface display. In certain embodiments, the sensor communicates with the drug delivery device in a closed-loop feedback system to modulate the activity of the drug delivery device, e.g., increasing, decreasing, maintaining, or ceasing the delivery of a therapeutic agent, e.g., insulin (in the case of a treatment for diabetes) or other medication, in response to analyte measurements determined by the sensor. In certain embodiments, the system comprises a closed-loop controlled system with wired or wireless data feedback.

In addition to the use of nanosensors as described above there are many physiologic parameters which would benefit from this type of long term real time data collection and analysis. The digitization of measurements such as blood pressure, heart rate, EKG, and respiration rate are some that could be monitored in this way.

Alternate RF Communications Between Sensor and Hand Held

In addition to the use of Blue Tooth protocol to provide wireless communicate between the Sensor and the Hand Held device, the use of NFC (Near Field Communication) protocol can possibly be used. The useful communication distance of NFC is inches instead of 30 feet for the Blue Tooth communications. This would require the Hand Held device to be held in close proximity to the sensor so that data could be transferred.

Using a Constant Current Source to Control the 460 nm LED Intensity

Because of the required sensitivity of the detector circuitry, small changes in detector current flow can be resolved. With reference to the circuit diagram of FIG. 13, when, e.g., the 460 nm LED(s) are first turned on, the PN junction inside the LED begins to heat up, which causes a change in the intensity of the light generated. A change in LED intensity causes measurement errors, and therefore, must be minimized or eliminated. In a preferred embodiment, the intensity of the 460 nm LED(s) is substantially or approximately constant (i.e., the intensity not change significantly). For example, the intensity of the LED can be controlled by maintaining a constant current through the device as illustrated in FIG. 13. In FIG. 13 a reference voltage is connected to the positive (“+”) pin of an opamp. The output of the opamp is connected to a transistor which controls the voltage on Resistor (R1) (held at voltage of AREF). The current flowing through the LED (“BLUE”) and R1 are the same. The opamp keeps its positive and negative inputs at the same voltage by keeping the transistor in the active zone. This keeps the current constant through R1 and the LED. Thus the opamp circuits are designed to hold the current through each LED constant which keeps the LED intensity constant.

Calculated A1c

The hemoglobin A1c test is a measure of a person's blood sugar control over the past 2 to 3 months. This is a periodic lab test which is performed on a blood sample provided by the patient. Typical A1c tests are snap shots every two to three months but the details of what is going on between each lab test are unknown. Thus, in certain aspects, the description provides a device/system as described herein for substantially continuous and/or periodic measurement of A1c levels. In additional, the description provides methods of using the device/system described herein for measuring A1c levels in a patient. In certain embodiments, the method comprises providing a device/system as described herein, wherein the device/system measures and calculates patient's glucose concentration at regular time intervals, e.g., every fractional second, second, minute, hour, day, week, month, year or combination thereof, for an extended period, such as several months or years, calculating a pseudo-A1c value for each corresponding time point, and graphically displaying the A1c data as, e.g., a curve, representing the patient's long term glucose levels. Through the device/system and methods as described herein, the long term control of the patient's glucose levels can be understood at a glance. At minimum, it would be easy to recognize if the A1c values are trending up or down.

In an additional embodiment, the description provides a method of monitoring an analyte in a patient non-invasively comprising the steps of administering an analyte-specific, light-emitting agent, e.g., a nanosensor, which emits light in the presence of the analyte; providing a sensor device comprising a plurality of LEDs, at least one photodiode configured to detect light emitted from the light-emitting agent, an SOC/microprocessor, and a transceiver for receiving/transmitting data wirelessly, wherein the sensor device is configured to detect and measure non-invasively the light emitted by the agent, calculate the amount or concentration of analyte present, and transmit the data to a display; detecting and measuring the intensity of light emitted by the analyte-specific light-emitting agent over a predetermined time course with the sensor device, wherein the sensor device calculates the amount or concentration of analyte present based on the intensity of light emitted by the light-emitting agent over the predetermined time course; transmitting the light emission data to a second processor having a display; displaying graphically the amount or concentration of the analyte detected by the sensor device over the predetermined time course. In a preferred embodiment, the analyte to be measured is glucose. In an additional embodiment, the sensor device calculates the amount of A1c as a function of glucose and transmits the data to a display.

Increasing Dynamic Range by LED Switching

As described previously, in certain exemplary embodiments a light source of 460 nm is created by LED(s) on each side of the 570 nm detector. The 570 nm wavelength of light creates a current in the detector which is scaled by the intensity of the 570 nm wavelength of light (this represents the concentration of glucose).

In general, the voltage output of the detector is amplified in the second stage of the detector circuit. This amplifier's gain is controlled by the value of a feedback resistor in an opamp circuit. A large dynamic range of measurement is classically obtained by using analog switches to enable different feedback resistor values. This changes the gain of the opamp circuit. This would provide high amplification to the 570 nm signal level when it was at low intensity and low amplification to the 570 nm signal level when it was at high intensity.

To overcome this issue, in certain embodiments, the device/system as described herein comprises two LED's that provide different intensities of 460 nm wavelength of light, respectively. For example, in certain embodiments, one LED may be biased with 5 mA of current (100% output) and the second LED may be biased at 2.5 mA (50% output). Then by alternating the power on and off to the LEDs at different times, a larger range of glucose concentrations can be measured. As an example, the 50% LED is turned ON, and a measurement can be taken. Then the 50% LED is turned OFF, and the 100% LED is turned ON, and another measurement is taken. Then both LEDs are turned ON which results in 150% illumination. This avoids the complications and errors of using analog switches in the feedback circuitry of the opamp.

In another aspect, the description provides a method for normalizing opamp circuit gain in an LED-based fluorescence detecting sensor device comprising the steps of: providing at least two LEDs that provide different intensities of 460 nm wavelength of light, wherein at least one LED is biased with about 5 mA of current, and at least one other LED is biased at 2.5 mA of current; alternating the power to the respective LEDs such that they are ON and OFF at different times; acquiring analyte measurements with (i) the 5 mA LED ON, and 2.5 mA LED OFF, (ii) the 5 mA LED OFF, and 2.5 mA LED ON, and (iii) both the 5 mA LED ON and the 2.5 mA LED ON. Varying the LED output in this was provides low, medium and high level of excitation providing a means for normalizing the signal strength to account for photobleaching or decay of nanosensor signal over time.

Heart Rate Monitor

In an alternate construction using different LEDs, detection of the pulsatile arterial blood flow can be used to measure and record a person's heart rate. By using a 660 nm or other wavelength LED, the same detector and high pass filter, the increase and decrease of arterial blood volume can be measured, from which the heart rate can be calculated. The measured time between two consecutive peaks can be used to calculate a heart rate at predetermined intervals, e.g., every minute.

The combination of continuous glucose concentration monitoring and continuous heart rate monitoring can provide additional physiological information which may lead to a better understanding of glucose consumption at diverse heart rates.

HIPPA Privacy Rules

The Standards for Privacy of Individually Identifiable Health Information (“Privacy Rule”) establishes, for the first time, a set of national standards for the protection of certain health information. The U.S. Department of Health and Human Services (“HHS”) issued the Privacy Rule to implement the requirement of the Health Insurance Portability and Accountability Act of 1996 (“HIPAA”). 1 The Privacy Rule standards address the use and disclosure of individuals' health information—called “protected health information” by organizations subject to the Privacy Rule—called “covered entities,” as well as standards for individuals' privacy rights to understand and control how their health information is used. Within HHS, the Office for Civil Rights (“OCR”) has responsibility for implementing and enforcing the Privacy Rule with respect to voluntary compliance activities and civil money penalties. A major goal of the Privacy Rule is to assure that individuals' health information is properly protected while allowing the flow of health information needed to provide and promote high quality health care and to protect the public's health and well-being. The Rule strikes a balance that permits important uses of information, while protecting the privacy of people who seek care and healing. Given that the health care marketplace is diverse, the Rule is designed to be flexible and comprehensive to cover the variety of uses and disclosures that need to be addressed.

The HIPAA laws must be addressed due to the personal medical information contained on the system website. As part of the design of this system, all the data handled will be encrypted from end-to-end. Once the ADC in the sensor collects the raw measured data it will encrypt it before storage in memory. It will have to be temporarily decrypted before it is charted on the Hand Held device or displayed on the website. Alternatively, if the Hand Held Device is password secured, the data is only encrypted while being transferred to and from the Hand Held Device and the Website. This way, all data being transferred over the Internet will be secure.

Using a PC browser to access to the website could be secure by using an HTTPS connection and a personal ID with a strong password. Also, the user could set authorization privileges for personal information to be accessed by health care providers or others.

Also, the use of higher security methods could be implemented to limit access. The use of a Personal Computer Smart Card (PCSC) reader and Smart Card Key with a secure PC browser could be used to contain decryption keys to limit access.

Those skilled in the art will recognize, or be able to ascertain using no more than routine experimentation, many equivalents to the specific embodiments of the invention described herein. Such equivalents are intended to be encompassed by the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. An optical sensor (optode) system comprising:
(i) at least one analyte-specific, light-emitting agent, which emits light in the presence of the analyte;
(ii) a sensor device configured to detect non-invasively the light emitted by the agent, the sensor device comprising a plurality of LEDs, at least one photodiode configured to detect light emitted from the light-emitting agent, an SOC/microprocessor, and transceiver to receive/transmit data wirelessly and, optionally, a photodiode light filter; and
(iii) a display, wherein the system is configured such that the sensor quantifies the in vivo levels or concentration of the analyte and displays the information graphically on the display.

2. The system of claim 1, wherein the sensor device comprises two LEDs.

3. The system of claim 1, wherein the LEDs are located near or in apposition to the photodiode or window thereof.

4. The system of claim 1, wherein the sensor device further comprises an OpAmp, an ADC, an encrypted memory or a combination thereof.

5. The system of claim 1, wherein the display is a hand-held device display or a computer data website or both.

6. The system of claim 1, wherein the analyte is selected from the group consisting of glucose, sodium, potassium, calcium, chloride, and a combination thereof.

7. The system of claim 1, wherein the system is further configured to acquire continuous real-time measurement of the analyte concentration.

8. The system of claim 1, further comprising a drug delivery device in communication with the sensor.

9. The system of claim 1, further comprising at least one additional sensor which detects or measures a vital sign in a subject, wherein the vital sign is blood pressure, heart rate, temperature or a combination thereof.

10. A method of monitoring an analyte in a patient non-invasively comprising the steps of:
administering an analyte-specific, light-emitting agent, which emits light in the presence of the analyte;
providing an optical sensor system comprising a sensor device comprising a plurality of LEDs, at least one photodiode configured to detect light emitted from the light-emitting agent, an SOC/microprocessor, a transceiver for receiving/transmitting data wirelessly, and a display, wherein the sensor device is configured to detect and measure non-invasively the light emitted by the agent, calculate the amount or concentration of analyte present;
detecting and measuring the intensity of light emitted by the analyte-specific light-emitting agent over a predetermined time course with the sensor device, wherein the sensor device calculates the amount or concentration of analyte present based on the intensity of light emitted by the light-emitting agent over the predetermined time course;
transmitting the light emission data to a second processor having a display;
displaying graphically the amount or concentration of the analyte detected by the sensor device over the predetermined time course.

11. The method of claim 10, wherein the sensor device comprises LEDs.

12. The method of claim 12, wherein the LEDs emit light at about 460 nm.

13. The method of claim 10, wherein the analyte is selected from the group consisting of glucose, sodium, potassium, calcium, chloride, and a combination thereof.

14. The method of claim 13, wherein the analyte is glucose.

15. A method for normalizing opamp circuit gain in an LED-based fluorescence detecting sensor device comprising the steps of:
providing at least two LEDs that provide different intensities of 460 nm wavelength of light, wherein at least one LED is biased with about 5 mA of current, and at least one other LED is biased at 2.5 mA of current;
alternating the power to the respective LEDs such that they are ON and OFF at different times; and
acquiring analyte measurements with (i) the 5 mA LED ON, and 2.5 mA LED OFF, (ii) the 5 mA LED OFF, and 2.5 mA LED ON, and (iii) both the 5 mA LED ON and the 2.5 mA LED ON.
